(19) United States
(12) Patent Application Publication
(10) Pub. No.: US 2010/0331534 A1
(43) Pub. Date: Dec. 30, 2010

(54) NUCLEIC ACID PURIFICATION METHOD

(75) Inventors: Manzer Khan, Union, NJ (US); Mubasher Dar, North Brunswick, NJ (US); Anabela Brito Gabriel, Union, NJ (US)

Correspondence Address: GE HEALTHCARE BIO-SCIENCES CORP.;PATENT DEPARTMENT, 101 CARNEGIE CENTER, PRINCETON, NJ, 08540, US

(73) Assignee: GE HEALTHCARE BIO-SCIENCES CORP., PISCATAWAY, NJ (US)

(21) Appl. No.: 12/667,747

(22) PCT Filed: Jul. 23, 2008
(86) PCT No.: PCT/US08/70837

§ 371 Date: Jan. 5, 2010

Related U.S. Application Data
(60) Provisional application No. 60/952,252, filed on Jul. 27, 2007.

(51) Int. Cl. C07H 1/06; C12N 5/07

(52) U.S. Cl. 536/23.1; 435/325

(57) ABSTRACT

The invention provides an modified method for the separation of nucleic acids from cells, comprising: generating an aqueous solution containing the nucleic acid by lysing the cells with a lysis solution including SDS and salt; and separating the nucleic acids of interest from other cellular components. The improvement includes adding a non-ionic detergent in the lysis solution such that SDS is not precipitated and no heating of the solution is required prior to cellular lysis. The preferred non-ionic detergents are the polysorbate family of compound, including TWEEN® 20. Also disclosed are composition and kit for performing the modified method.

### CROSS-REFERENCE TO RELATED APPLICATIONS

This application is a filing under 35 U.S.C. §371 and claims priority to international patent application number PCT/US2008/070837 filed Jul. 23, 2008, published on Feb. 5, 2009, as WO 2009/018034, which claims priority to U.S. provisional patent application No. 60/952,252 filed Jul. 27, 2007; the disclosure of which is incorporated herein by reference in its entirety.

### FIELD OF THE INVENTION

This invention relates generally to methods for the isolation of nucleic acids from contaminating cellular components. In particular, the invention relates to improved processes for purification of nucleic acids, by providing a lysis solution including a non-ionic detergent such that a pre-heating step of the solution is eliminated. The invention further provides compositions and kits for performing the improved nucleic acid purification process.

### BACKGROUND OF THE INVENTION

The last three decades has seen considerable effort in the development of improved methods for the isolation and purification of nucleic acids from biological sources. This has been due mainly to the increasing applications of nucleic acids in the medical and biological sciences. Genomic DNA isolated from blood, tissue or cultured cells has several applications, which include PCR, sequencing, genotyping, hybridization and southern blotting. Plasmid DNA has been utilized in sequencing, PCR, in the development of vaccines and in gene therapy. Isolated RNA has a variety of downstream applications, including blot hybridization, in vitro translation, cDNA synthesis and RT-PCR.

The analysis and in vitro manipulation of nucleic acids is typically preceded by a nucleic acid isolation step in order to free the nucleic acid from unwanted contaminants which may interfere with subsequent processing procedures. For the vast majority of procedures in both research and diagnostic molecular biology, extracted nucleic acids are required as the first step. In a typical DNA extraction protocol, cells or homogenized tissue samples containing the nucleic acid of interest are harvested and lysed using standard methods, for example using enzymes such as Proteinase K and lysozyme; detergents, such as SDS, or using other chemicals such as sodium hydroxide, guanidium isothiocyanate, etc. (See for example, Sambrook and Russell, Molecular Cloning—A Laboratory Manual 3nd edition 6.4 (New York: Cold Spring Harbor Laboratory 2001)). Following removal of the cellular debris, the crude lysate is treated with organic solvents such as phenol/chloroform to extract proteins. RNA may be removed or reduced if required by treatment of the enzymes such as RNAse. However, the presence of contaminants such as salts, phenol, detergents and the like can interfere with many downstream manipulations for which the nucleic acid is intended.

Currently several procedures are available for the chromatographic purification of DNA (genomic and plasmid) and RNA, for example, by employing silica based membrane purification, size exclusion chromatography, reversed phase chromatography, gel filtration, magnetic bead based purification, or ion-exchange chromatography. These procedures have been developed into commercial products in the form of kits, including the QIAAMP® DNA Mini Kit for genomic DNA isolation (#51304, Qiagen Inc., Valencia, Calif.).

High salt, SDS and guanidine buffers are commonly used in molecular biology and in nearly all microarray protocols. In particular, SDS and high salt together form ubiquitous buffers that are used in many applications such as sample preparations, cell/tissue/blood lysis, southern/northern/western blotting, as well as array hybridization. The high salt aids in binding nucleic acids to the silica based matrix or other resins, whereas the SDS aids in efficient cell lysis. While the efficacy of SDS and salt generally increases with concentration, the actual concentrations of the two are limited by the solubility of SDS which precipitates in high salt (˜2M NaCl). To circumvent this problem, a pre-heating step is required to bring SDS in solution that can last up to 2-3 hours. For example, in microarray protocols, pre-heating is done for several hours prior to stringency washing steps. For sample preparation methods involving cellular lysis, high SDS and salt concentrations have not been employed in lysis step as pre-heating step is cumbersome demanding extra time to process samples and the possible interference of lysis mixture (lysate) in purification due to column clogging.

There is a need for an improved solution for molecular biology applications, which increases the solubility of SDS in high salt buffer, therefore a higher amount of SDS can be included, yet eliminates the need for a pre-heating step before the application.

### BRIEF DESCRIPTION OF THE INVENTION

It is surprisingly found that the inclusion of certain non-ionic detergents in even small amounts (0.5-2%) increases the solubility of SDS in high salt buffers thus permitting higher concentrations of SDS and salt concentrations to be utilized. It also eliminates the pre-heating step required for solutions without the non-ionic detergent, to bring SDS back in solution prior to use.

Thus, in a first aspect the present invention provides a method for the separation and/or purification of a nucleic acid from cells, comprising: a) generating an aqueous solution containing the nucleic acid by lysing the cells with a lysis solution including SDS and salt; and b) separating the nucleic acid from other cellular components; characterized in that a non-ionic detergent is included in the lysis solution so that SDS is not precipitated and no pre-heating of the solution is required prior to step a). Preferably, the non-ionic detergent is a polysorbate. Most preferably, the non-ionic detergent is TWEEN® 20.

In a second aspect, the invention provides a composition for the lysis of cells including a salt buffer, SDS and a non-ionic detergent. Preferably, the non-ionic detergent is a polysorbate. Most preferably, the non-ionic detergent is TWEEN® 20.

The invention additionally provides a kit for the separation and/or purification of nucleic acid from cells, which kit includes a lysis solution for lysing cells, including a salt buffer, SDS and a non-ionic detergent. Preferably, the non-ionic detergent in the kit is a polysorbate. Most preferably, the non-ionic detergent is TWEEN® 20. The kit additionally includes a user manual.

Further aspects and uses of the current invention will become apparent from a consideration of the ensuing description.

### BRIEF DESCRIPTION OF THE DRAWINGS

FIG. 1 shows real time PCR amplification results obtained from the genomic DNA samples from rat liver, with very similar amplification profiles observed among the samples.

FIG. 2 shows comparison of restriction enzyme (BamHI) digested and un-digested genomic DNA that was purified from rat liver samples.

### DETAILED DESCRIPTION OF THE PREFERRED EMBODIMENTS

High salt, SDS and guanidine buffers are commonly used in molecular biology and in nearly all microarray protocols. In particular, SDS and high salt together form ubiquitous buffers that are used in many applications such as sample preparations, cell/tissue/blood lysis, southern/northern/western blotting, as well as array hybridization.

While the efficacy of SDS and salt generally increases with concentration, the actual concentrations of the two are limited by the solubility of SDS which precipitates in high salt (˜2M NaCl). To circumvent this problem, a pre-heating step is required to bring SDS in solution that can last up to 2-3 hours. For sample preparation methods, high salt and high SDS concentrations are not combined to avoid a preheating step which can slow down the speed of purification.

We surprisingly discovered that certain non-ionic detergents, when mixed with SDS, form detergent complexes increasing the solubility of SDS in aqueous solution. The use of these non-ionic detergents in small concentration (0.5-2%) increases the solubility of SDS in high salt or guanidine buffers thus permitting higher concentrations of SDS to be utilized. Because SDS is solubilised, the pre-heating step is not needed prior to use.

We also show that the inclusion of the non-ionic detergents does not affect the functionality of the buffer solution for genomic DNA isolation.

Thus, the present invention first provides a method for the separation and/or purification of a nucleic acid from cells, comprising: a) generating an aqueous solution containing the nucleic acid by lysing the cells with a lysis solution including SDS and salt; and b) separating the nucleic acid from other cellular components; characterized in that a non-ionic detergent is included in the lysis solution so that SDS is not precipitated and no pre-heating of the solution is required prior to step a).

The term “nucleic acid” as used herein refers to any DNA or RNA molecule, or a DNA/RNA hybrid, or mixtures of DNA and/or RNA. The term “nucleic acid” therefore is intended to include genomic or chromosomal DNA, plasmid DNA, total RNA and mRNA. The process according to the present invention is particularly suitable for the preparation and/or purification of genomic DNA derived from complex mixtures of components derived from cellular and tissue samples from any recognised source, including normal and transformed cells, with respect to species (e.g. plants, animals, bacteria), tissue source (e.g. brain, liver, lung, heart, kidney skin, muscle) and cell type (e.g. epithelial, endothelial, blood).

Furthermore, the present method is suitable for the preparation and/or purification of genomic DNA having a size of from about 0.1 kilo-bases to about 200 kilo-bases, or of plasmid DNA, cosmid, BAC or YAC. The present invention is useful for purifying plasmid DNA and cosmid DNA, in particular for downstream applications in molecular biological research, such as cloning and sequencing, gene therapy and in diagnostic applications both in vivo and in vitro.

Lysis of cells generally is performed using a high salt buffer including an ionic detergent such as SDS, in the presence of a proteinase. A common composition of the lysis solution includes sodium chloride, SDS, Tris and EDTA. For genomic DNA isolation, the cell lysate is treated with a guanidine solution for the extraction of DNA from the other components (e.g., proteins, etc).

The use of certain non-ionic detergents increases the solubility of the ionic deteregent (i.e., SDS), enabling a higher amount of the ionic detergent in the solution, also eliminates the need for preheating the solution prior to an application. It is noted that while certain non-ionic detergents work well, others are less effective. For example, while TWEEN® 20 works well, TRITON® X-100 and NP 40 do not perform satisfactorily. The polysorbate family of chemicals are a preferred group of non-ionic detergents.

TWEEN® 20 is a frequently used member of the polysorbate family. TWEEN® 20 is a polyoxyethylene sorbitol ester, with a calculated molecular weight of 1,225 Daltons, assuming 20 ethylene oxide units, 1 sorbitol, and 1 lauric acid as the primer fatty acid. It is a non-ionic detergent widely used in biochemical applications, including as an emulsifying agent for the preparation of stable oil-in-water emulsions, as a blocking agent for membrane based immunoassays, and a solubilizing agent for membrane proteins. TWEEN® 20 has also been used for lysing mammalian cells at a concentration of 0.05-0.5%.

The use of TWEEN® 20 at even a very high concentration (30%), as a SDS solubilisation agent in a sodium chloride salt buffer, allows the isolation of genomic DNA from cell samples. The use of as low as 0.5% of the agent is effective. TWEEN® 20 is also effective in solubilising SDS in a potassium chloride solution, albeit a higher concentration of TWEEN® 20 is needed. The inclusion of TWEEN® 20 simplifies the protocol by eliminating the pre-heating step, and enables a higher amount of SDS to be used in the lysis solution. Moreover, the yield and quality of genomic DNA isolated are comparable to that without TWEEN® 20.

The effect of non-ionic detergent, such as TWEEN® 20, in solubilising SDS in a salt (including guanidine) buffer, as well as the amount needed for each SDS/salt buffer combination, can be easily established. Further, any adverse effect the non-ionic detergent might have for the downstream application can be tested following standard procedures. In this sense, the use of the finding is not limited to the isolation of genomic DNA, but many molecular biology and microarray/hybridization experiments. In addition to sodium and potassium salt buffers, TWEEN® 20 at varying concentrations is also effective for buffers containing calcium chloride, ammonium sulphate, as well as guanidinium HCl. We also found that TWEEN® 20 is effective in an agmatine (i.e., 2 aminobutyl guanidine) buffer.

TWEEN® 20 is a tradename for polyethylene glycol sorbitan monolaurate 20 (alternatively polyoxyethylenesorbitan monolaurate 20). Other commercially available polysorbates include TWEEN® 40, TWEEN® 60 and TWEEN® 80 (Sigma-Aldrich, St. Luois, Mo.). Any of these and other related chemicals is effective as a replacement of TWEEN® 20 for the purpose of the invention.

The invention also provides a method for improving the signal to noise ratio for nucleic acid hybridization assays, such as microarray analysis, Southern and Northern blotting assays. Hybridization and wash buffers for nucleic acid hybridization assays contain high salt and SDS, are commonly referred to as SSC/SDS buffers. These buffers contain sodium chloride and SDS which precipitate at high salt concentration. Prior heating of the buffers is required to remedy this effect. A non-ionic detergent (e.g., TWEEN® 20) when added at low concentration such as 0.5-2% prevents SDS precipitation facilitating higher concentrations of SDS and eliminating the pre-heating step. An increased amount of SDS lowers the background hybridization signal. DNA hybridization assays, such as microarray assays, Southern and Northern blotting assays all benefit from a reduced background.

In a second aspect, the invention provides a composition including a salt buffer, SDS and a non-ionic detergent. Preferably, the non-ionic detergent is polysorbate. More preferably, the non-ionic detergent is TWEEN® 20. The inclusion of a non-ionic detergent such as TWEEN® 20 increases the solubility of SDS in a high salt solution. This composition is useful for the lysis of cells for isolation of cellular components such as genomic DNA, RNA, plasmid DNA and proteins. The composition is also useful for nucleic acid hybridization assays, to increase the signal to noise ratio. Additionally, a solution that does not precipitate at room temperature finds its use in sample preparation using miniature capillary-based devices, preventing system clogging. A TWEEN® 20/SDS/salt buffer is useful for a miniature sample preparation device for the extractions and analysis of cellular components such as nucleic acids and proteins.

In a third aspect, the invention provides a kit for the separation and/or purification of nucleic acid from a cellular sample, the kit comprising a cellular lysis solution including a salt buffer, SDS and a non-ionic detergent; and a user manual. Suitably the non-ionic detergent is polysorbate. Preferably, the non-ionic detergent is TWEEN® 20.

Other features and advantages of the invention will be apparent from the following examples and from the claims.

### EXAMPLES

The following examples serve to illustrate the DNA purification processes according to embodiments of the present invention and are not intended to be limiting.

### 1. Buffers and Protocols Used in the Examples

### Detailed Composition of Solutions Used in the Protocols

PBS: 137 mM NaCl; 2.7 mM KC1; 4.3 mM Na₂HPO4; 1.47 mM KH₂PO4. Adjust to a final pH of 7.4.
Tissue and Cell Lysis Buffer: 2 M NaCl, 1.2% SDS, 12 mM EDTA, 24 mM Tris-HCl, pH8.0; with 2% TWEEN® 20 unless otherwise specified.
10 μl of proteinase K: 20 mg/ml resuspended in water.
20 mg/ml RNase solution made from a stock.
Extraction Buffer: 50 mM Tris, 10 mM EDTA, 7M Guanidine-HCl, 5% TWEEN® 20, set to pH7 with hydrochloric acid

Wash Buffer: 10 mM Tris HCl, pH7, 1 mM EDTA, pH8.0, and 80% ethanol.

TE: 10 mM Tris HCl and 0.5 mM EDTA, pH8.

Isolation of genomic DNA from tissue samples
1. Using a sterile blade to slice tissue, weigh out 5-50 mg of animal tissue and transfer it to the bottom of a 2 ml microcentrifuge tube. Keep the tubes on ice until step 2 below.
   When working with mouse tails, it is recommended to first freeze tissue by pouring liquid nitrogen in to a mortar with thin tail slices. The frozen tissue should then be crushed into very small pieces with a pestle and transferred into a microcentrifuge tube for homogenization.
2. Wash the tissue slice with 1 ml of PBS. Centrifuge at maximum speed for 1-2 minutes. Discard supernatant by aspiration or by inverting the tube taking care not to disturb the sample.
3. Add 50 μl of PBS to each microcentrifuge tube containing slice(s) of animal tissue.

Homogenize the tissue completely into solution.
4. Add 50 μl of Tissue and Cell lysis Buffer.
5. Pipette 10 μl of proteinase K into microcentrifuge tube. Vortex the tube at maximum speed for 15 seconds.
6. Incubate the tube(s) at 56° C. for 30-60 minutes.
   During the incubation step, fill several microcentrifuge tubes with TE buffer and heat to 70° C. in a heat block. Pre-warm TE is needed for the final elution step.
   7. Optional step. Add 5 μl of 20 mg/ml RNase solution. Incubate at room temperature for 15 minutes to isolated RNA-free genomic DNA.
8. Pipet 500 μl of Extraction Buffer to each tube. Vortex at maximum speed for 15 seconds. Incubate the tubes at room temperature for 10 minutes.
9. Remove the desired number of silica columns with collection tube and place them in a rack. Apply each sample to separate columns and spin at 11,000×g for 1 minute.
10. Discard the flow through and add 500 μl of Extraction Buffer. Centrifuge at 11,000×g for 1 minute. Discard the collection tube with the flow through. Transfer the column to a new collection tube.
11. Apply 500 μl of Wash Buffer and spin at maximum speed for 3 minutes. Discard the collection tube and transfer the columns to a new clean microcentrifuge tube.
12. Add 200 μl of pre-warmed TE (at 70° C.) directly on top of the column. Incubate the columns at room temperature for 1 minute.
13. Spin at 11,000×g for 1 minute to collect the purified genomic DNA.
   A second elution step will increase yield by ˜15-20%.
      Isolation of Genomic DNA from Mammalian Cell Lines
      This protocol allows genomic DNA isolation from up to 5×10⁶ cultured cells. Attached cells should be trypsinized and washed once with PBS prior to DNA extraction.
1. Transfer 1-5×10⁶ cultured cells to microcentrifuge tube(s). Centrifuge cells at 5000 rpm for 1 min. A visible cell pellet will appear at the bottom of the tube.
2. Resuspend the cell pellet with 1 ml of PBS to remove traces of trypsin and/or serum.

Centrifuge at 5000 rpm for 1 min. A visible cell pellet will appear at the bottom of the tube.
3. Add 40 μl of PBS and resuspend cells using a pipette.
4. Add 100 μl of Tissue and Cell Lysis Buffer and resuspend cells completely by vortexing for 15 seconds.
5. Add 10 μl of proteinase K (20 mg/ml) to each sample. Vortex again for 15 seconds.
6. Incubate samples at 56° C. for 15 minutes followed by 70° C. for 2 minutes.
   During the incubation step, fill several microcentrifuge tubes with TE buffer and heat to 70° C. in a heat block. Pre-warm TE is needed for the final elution step.
7. Optional step: Add 5 μl of RNase A (20 mg/ml) to each sample and incubate at RT for 15 min.
8. Add 500 μl Extraction Buffer to each tube and leave at RT for 10 min.
9. Remove the desired number of silica columns with collection tube and place them on rack. Apply sample to column and spin at 11,000×g for 1 min. Discard flow through. The entire sample should flow through the column. If any of the columns clog, spin them at maximum speed for 15-30 seconds to clear the residue before proceeding with wash steps below.
10. Apply 500 μl of Extraction Buffer to column. Centrifuge at 11,000×g for 1 min. Discard the collection tube with flow through. Transfer the column to a new collection tube.
11. Apply 500 μl Wash solution and spin at max speed for 3 min.
12. Add 200 μl of pre-warmed TE (at 70° C.) directly on top of the column. Incubate the columns at room temperature for 1 minute.
13. Spin at 11,000×g for 1 minute to collect the purified genomic DNA. A second elution step will increase yield by ˜15-20%.
   2. Optimization of the lysis solution for genomic DNA isolation

SDS in the lysis solution (2 M NaCl, 1.2% SDS, 12 mM EDTA, 24 mM Tris-HCl, pH8.0) becomes insoluble at room temperature. A preheating step is required to bring SDS into solution, prior to the addition into a sample for cellular lysis. We discovered that the inclusion of a small amount of a non-ionic detergent (e.g., TWEEN® 20) can increase the solubility of SDS such that no precipitation is observed in the lysis solution, and no preheating is needed prior to cell lysis. The yield and quality of the nucleic acid isolated is comparable or better than that without the non-ionic detergent.

In an effort to find an optimal lysis solution for genomic DNA purification, various TWEEN® 20 levels were tested, in combination with the lysis solution. Genomic DNA was isolated according to the protocol described in the previous section. Addition of 2% TWEEN® 20 in the lysis solution not only keeps SDS in solution, but also provides uncompromised yield of genomic DNA (Table 1).

TABLE 1
Lysis buffer and TWEEN ® 20 combination for
genomic DNA extraction from 10 mg of rat liver.

| Lysis Buffer | Genomic DNA yield (μg) | Genomic DNA purity* |
|---|---|---|
| without TWEEN ® 20 | 12.4 | 1.94 |
| with 10% TWEEN ® | 6.2 | 1.92 |
| with 30% TWEEN ® | 5.3 | 1.93 |
| with 2% TWEEN ® | 12.9 | 1.94 |

*DNA purity was determine by measuring ratio of absorbance values at 260 and 280 nm using the Biotek UV-VIS plate reader.

3. Isolation of Genomic DNA from Tissue Samples

We tested a variety of tissue sources for the performance of the modified lysis solution containing 2% TWEEN® 20. Multiple samples were processed to assess the consistency of the protocol. The purity of the product was measured by UV spectrophotometry and by gel analysis. The genomic DNA obtained was also evaluated in downstream applications such as real time PCR, and restriction digestion. Our results show that the modified protocol as described above works well for all the tissue sources tested.

Table 2 presents genomic DNA isolation results obtained from rat liver tissue, using both the current protocol and that from a Qiagen kit (QIAAMP® DNA Mini Kit, Qiagen Inc., Valencia, Calif.). We measured the absorbance values at 260 and 280 nm using the Biotek UV-VIS plate reader, which shows that the modified protocol produces consistent, high quality genomic DNA from liver tissue. While both protocols generate high quality DNA, our modified protocol consistently produces 30-40% more genomic DNA than the QIAAMP® kit from rat liver.

TABLE 2
Genomic DNA isolation from 20 mg of rat liver. Data below
is obtained from 54 repeat experiments for each of the current
protocol and 36 repeat experiments for QIAAMP ®.

| Protocol and kit used | DNA Yield (μg) |
|---|---|
| Current protocol, 2 Wash | 20.63 ± 4.37 |
| Current protocol, 1 Wash | 21.71 ± 4.09 |
| QIAAMP ® DNA mini kit | 13.80 ± 4.47 |

The quality of the purified genomic DNA was assessed by real time PCR assays. Real time PCR reactions were set up using 100 ng of purified rat liver genomic DNA per sample (n=36 from the modified protocol, n=4 for QIAAMP® kit) using the PuReTaq READY-TO-GO™ (RTG) PCR beads (GE Healthcare, Piscataway, N.J.) in the presence of GELSTAR™ dye (Cambrex, Baltimore, Md.) using primers specific for the GAPDH gene.

### Real-Time PCR Reaction

Dilute genomic DNA template prepared from rat liver tissue to 20 ng/ul in water (Using 100 ng of template per reaction).

| | |
|---|---|
| Component | Amount added per reaction |

| | | |
|---|---|---|
| PuReTaq RTG PCR bead | 1 | bead |
| water | 17 | μl |
| GAPDH forward PCR primer [5 μM] | 1 | μl |
| GAPDH reverse PCR primer [5 μM] | 1 | μl |
| 1:1000 GELSTAR ™ dye | 1 | μl |
| genomic DNA [20 ng/ul] | 5 | μl |
| Final Reaction volume | 25 | μl |

The amplification was monitored on an ABI7900HT Fast Real-Time PCR System (Applied Biosystems Inc., Foster City, Calif.), following these cycling conditions:

95° C., 15 minutes

| | |
|---|---|
| 95° C., 15 seconds | 40 cycles |
| 54° C., 30 seconds | |
| 72° C., 30 seconds | |
| 95° C., 15 seconds | Default ABI7900 instrument |
| 60° C., 15 seconds | dissociation curve |
| 95° C., 15 seconds | |

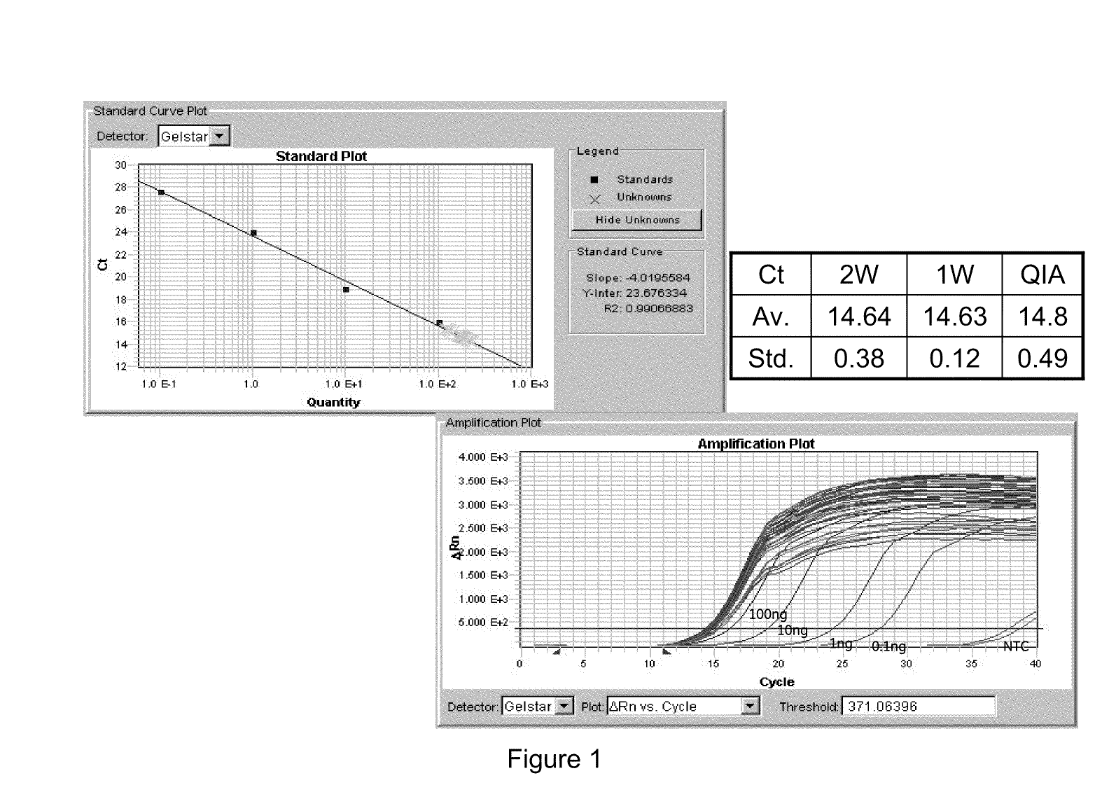

FIG. 1 shows real time PCR amplification results obtained. The amount of signal correlates with amplification of the GAPDH gene. The point at which signal rises above background threshold is defined as Ct value for the amplification. 2W: column washed twice with the wash solution prior to elution. 1W: column washed once prior to elution. QIA: genomic DNA isolated using the QIAAMP® kit. All the samples tested show very similar amplification profiles.

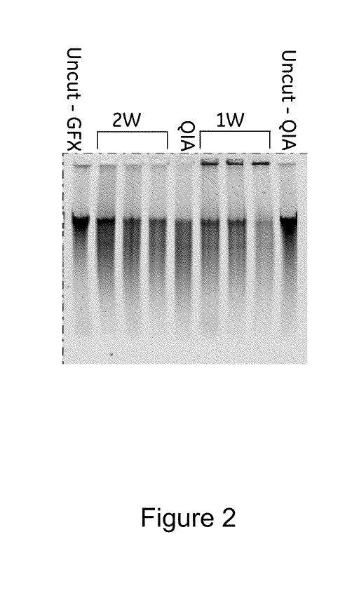

The purified genomic DNA was also subjected to restriction enzyme digest using BamHI. Purified genomic DNA (2 ug) was digested with the enzyme under standard conditions. The digested sample was analyzed on an agarose gel side-by-side with un-digested sample DNA (uncut—GFX), and a sample obtained using the QIAAMP® kit (QIA; and uncut—QIA). The gel image shows that the genomic DNA isolated after a second wash of the column was completely digested (FIG. 2, 2W), while those underwent a single wash was not as well digested (FIG. 2, 1W).

Table 3 presents genomic DNA isolation results obtained from rat kidney and mouse tail. The absorbance values at 260 and 280 nm were measured using the Biotek UV-VIS plate reader. It shows that the modified protocol produces consistent, high quality results in isolating genomic DNA from these tissues.

TABLE 3
Genomic DNA isolation from 15 mg of rat kidney
(n = 4) or mouse tail (n = 6).

| Tissue type | Yield (μg) | Purity (260/280) |
|---|---|---|
| Rat Kidney | 19.56 ± 1.7 μg | 1.81 |
| Mouse Tail | 11.7 ± 1.7 μg | 1.84 |

The purity of the sample was also examined by an agarose gel analysis, restriction digest and real time PCR assay. It demonstrates that the genomic DNA isolated is pure and without RNA contamination.

4. Isolation of Genomic DNA from Cell Cultures

Cultured cells were also tested for the performance of the modified lysis solution containing 2% TWEEN® 20. Multiple samples were processed, each with a different amount of input cell mass. The purity of the product was assessed by UV spectrophotometry and by gel analysis. The genomic DNA obtained was also evaluated in downstream applications such as real time PCR, and restriction digestion. Our results show that the modified protocol as described above works well for cultures cell as well.

Table 4 presents genomic DNA isolation results obtained from cultured CHO cells. It shows that the modified protocol produces consistent, high quality results in isolating genomic DNA from cultured cells.

TABLE 4
Genomic DNA yield and purity data from CHO cells.

| Cell number | DNA Yield (μg) | purity |
|---|---|---|
| 5 × 10⁶ | 13.89 | 1.84 |
| 3 × 10⁶ | 8.79 | 1.85 |
| 1 × 10⁶ | 5.22 | 1.86 |
| 0.5 × 10⁶ | 3.62 | 1.88 |
| 3 × 10⁵ | 2.57 | 1.88 |
| 1 × 10⁵ | 1.52 | 1.97 |

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

1. A method for the separation of a nucleic acid from cells, comprising:
a) generating an aqueous solution containing the nucleic acid by lysing said cells with a lysis solution including SDS and salt; and
b) separating said nucleic acid from other cellular components;
the improvement comprises adding a non-ionic detergent in said lysis solution such that SDS is not precipitated and no heating of said solution is required prior to step a).

2. The method of claim 1, wherein said non-ionic detergent is a polysorbate.

3. The method of claim 2, wherein said polysorbate is TWEEN® 20.

4. The method of claim 3, wherein said TWEEN® 20 is at a concentration of about 0.5% to about 30%.

5. The method of claim 3, wherein said TWEEN® 20 is at a concentration of about 2%.

6. The method of claim 1, wherein said salt is sodium salt, potassium salt, calcium salt, ammonium salt, guanidinium HCl or agmatine.

7. The method of claim 1, wherein said salt is sodium chloride or potassium chloride.

8. The method of claim 1, wherein the nucleic acid is genomic DNA.

9. The method of claim 1, wherein the nucleic acid is RNA.

10. The method of claim 1, wherein the nucleic acid is plasmid DNA.

11. A composition for the lysis of cells including a salt buffer, SDS and a non-ionic detergent.

12. The composition of claim 11, wherein said non-ionic detergent is polysorbate.

13. The composition of claim 12, wherein said polysorbate is TWEEN® 20.

14. The composition of claim 13, wherein said TWEEN® 20 is at a concentration of about 0.5% to about 30%.

15. The composition of claim 13, wherein said TWEEN® 20 is at a concentration of about 2%.

16. The composition of claim 11, including 2 M sodium chloride, 1.2% SDS, 12 mM EDTA, 24 mM Tris-HCl, pH8.0 and 2% TWEEN® 20.

17. A kit for the separation and/or purification of nucleic acid from cells, comprising: a cellular lysis solution including a salt buffer, SDS and a non-ionic detergent; and a user manual.

18. The kit of claim 17, wherein said non-ionic detergent is polysorbate.

19. The kit of claim 17, wherein said non-ionic detergent is TWEEN® 20.

20. The kit of claim 17, wherein said lysis solution includes 2 M sodium chloride, 1.2% SDS, 12 mM EDTA, 24 mM Tris-HCl, pH8.0 and 2% TWEEN® 20.
